(19) United States
(12) Patent Application Publication
(10) Pub. No.: US 2011/0217379 A1
(43) Pub. Date: Sep. 8, 2011

(54) MAGNETIC NANOMATERIALS AND METHODS FOR CHEMOEMBOLISATION

(75) Inventors: Yixiang Wang, Shatin (CN); Ken Cham-Fai Leung, Ma On Shan (CN); Chris Hon Ki Cheng, Ma On Shan (CN); Ling Qin, Taipo (CN)

(73) Assignee: Davis LLP, Vancouver (CA)

(21) Appl. No.: 13/040,873

(22) Filed: Mar. 4, 2011

Related U.S. Application Data
(60) Provisional application No. 61/311,647, filed on Mar. 8, 2010.

(51) Int. Cl. A61K 9/50; A61K 31/745; A61K 31/765; A61P 35/00; A61K 9/26; B82Y 5/00

(52) U.S. Cl. 424/486; 424/484; 424/78.31; 427/2.14; 977/905

(57) ABSTRACT

There are disclosed magnetic nanoparticles and embolisation compositions comprising the nanoparticles. There are also disclosed methods to make the nanoparticles and embolisation compositions and methods to deliver therapeutic agents to a subject.

### CROSS-REFERENCES TO RELATED APPLICATIONS

This application claims the benefit of U.S. Provisional Application No. 61/311,647, filed Mar. 8, 2010, the contents of which are hereby incorporated by reference.

### BACKGROUND OF THE INVENTION

The present invention relates to magnetic nanoparticles and embolisation compositions comprising the nanoparticles.

### RELATED PRIOR ART

U.S. Pat. No. 6,123,920 to Wolfgang H. H. Gunther et al. issued Sep. 26, 2000 discloses magnetic resonance contrast media containing composite nanoparticles.
US 2007/0154397 to Wen Hsiang Chang et al. published Jul. 5, 2007 discloses a thermosensitive nanostructure for hyperthermia treatment.
U.S. Pat. No. 6,962,685 to Shouheng Sun, issued Nov. 8, 2005, discloses a method and structure for making magnetite nanoparticle materials.
Related China patents from the State Intellectual Property Office of the P. R. China: F. Tang et al. 03156712.6 issued on Mar. 16, 2005; 200310122436.X issued on Jun. 29, 2005; and 200610081252.7 issued on Nov. 28, 2007 (In Chinese)
Laurent A. Microspheres and nonspherical particles for embolization. Tech Vasc Intery Radiol. 2007; 10:248-56;
Chu J P, Chen W, Li J P, Zhuang W Q, Huang Y H, Huang Z M, Yang J Y. Clinicopathologic Features and Results of Transcatheter Arterial Chemoembolization for Osteosarcoma. Cardiovasc Intervent Radiol. 2007; 30:201-6;
Vogl T J, Gruber T, Balzer J O, Eichler K, Hammerstingl R, Zangos S. Repeated Transarterial Chemoembolization in the Treatment of Liver Metastases of Colorectal Cancer: prospective study. Radiology. 2009; 250:281-9:
Owen R J. Embolization of Musculoskeletal Tumors. Radiol Clin North Am. 2008; 46:535-43
Udayasankar U, Chamsuddin A, Mittal P, Small W C. Diagnostic Imaging and Image-guided Interventions of Hepatobiliary Malignancies. Cancer Treat Res. 2008; 143:199-228
Cho K, Wang X, Nie S, Chen Z G, Shin D M. Therapeutic Nanoparticles for Drug Delivery in cancer. Clin Cancer Res. 2008; 14:1310-6.
Wang X, Yang L, Chen Z, Shin D M. Application of Nanotechnology in Cancer Therapy and Imaging CA Cancer J Clin 2008; 58; 97-110
Wang Y X, Hussain S M, Krestin G P. Superparamagnetic Iron Oxide Contrast Agents: Physiochemical Characteristics and Applications in MR Imaging. European Radiology 2001, 11, 2319-2331.
Corot C, Robert P, Idée J M, Port M. Recent advances in iron oxide nanocrystal technology for medical imaging. Adv Drug Deliv Rev. 2006; 58:1471-504
Alexiou C; Arnold W; Klein R J; Parak F G; Hulin P; Bergemann C; Erhardt W; Wagenpfeil S; Lübbe A S. Locoregional Cancer Treatment with Magnetic Drug Targeting. Cancer Research 2000, 60, 6641-6648.
Wilson M W, Kerlan R K Jr, Fidelman N A, Venook A P, LaBerge J M, Koda J, Gordon R L. Hepatocellular Carcinoma: Regional Therapy with a Magnetic Targeted Carrier Bound to Doxorubicin in a Dual MR Imaging/Conventional Angiography Suite—Initial Experience with Four Patients. Radiology 2004; 230:287-93
Lübbe A S; Bergemann C; Riess H; Schriever F; Reichardt P; Possinger K; Matthias M; Dorken B; Herrmann F; Gurtler R; Hohenberger P; Haas N; Sohr R; Sander, B; Lemke A J; Ohlendorf D; Huhnt W; and Huhn D. Clinical Experiences with Magnetic Drug Targeting: a Phase I Study with 49-Epidoxorubicin in 14 Patients with Advanced Solid Tumors. Cancer Research 1996, 56, 4686-4693.
Just R; Hoh C; Vogl T; Neese P; Doemeny J; Schechter M; Varney R; Stanton W; Schiemann M; Goldfarb P. A Phase I/II Single Arm Trial to Determine the Safety, Tolerability, and Biological Activity of Intrahepatic Delivery of Doxorubicin Hydrochloride Adsorbed to Magnetic Targeted Carriers (MTX-DOX) in Patients with Metastatic Tumors in the Liver. European Journal of Cancer (Supplement) 2003, 1, S292-S293.
Bendszus M, Klein R, Burger R, Warmuth-Metz M, Hofmann E, Solymosi L. Efficacy of Trisacryl Gelatin Microspheres Versus Polyvinyl Alcohol Particles in the Preoperative Embolization of Meningiomas. AJNR Am J. Neuroradiol. 2000; 21:255-61
Worthington-Kirsch R, Fueredi G, Goodwin S, Machan L, Niedzwiecki G, Reidy J, Spies J, Walker W. Polyvinyl Alcohol Particle Size for Uterine Artery Embolization. Radiology 2001; 218:605-6.
U.S. Pat. No. 5,160,725 issued Nov. 3, 1992 and WO-94/21240, both to Pilgrimm, disclose physiologically tolerated dispersions of superparamegnetic particles and reactive stabilizer bonded to the surface of the superparamagnetic particles.
PCT/GB94/02097 Filed Sep. 27, 1994, Fahlvik et al. discloses superparamagnetic nanoparticles for use as contrast agents in magnetic resonance imaging.
U.S. Pat. No. 5,464,696 issued Nov. 7, 1995 to Bracco discloses magnetite particles suitable for injection into the blood stream of patients. The particles comprise an iron oxide core and an organic chemical shell surrounding the core.
U.S. Pat. No. 4,904,479 issued Feb. 27, 1990 to 11 um discloses a drug delivery system comprising a suspension of colloidal particles, each coated with a hydrophilic coat.
University of California—Santa Cruz (2009, Mar. 28). Hollow Gold Nanospheres Show Promise For Biomedical And Other Applications. http://www.sciencedaily.com/releases/2009/03/090322154415.htm.
Hollow Nanocrystals and how to Mass Produce Them (2004, May 28) Science Beat, Berkeley Lab. http://www.lbl.gov/Science-Articles/Archive/sb/May-2004/02-MSD-hollow-nanocrystals.html.

### BRIEF SUMMARY OF THE INVENTION

In an embodiment there is disclosed an embolization composition comprising a biodegradable matrix encapsulating at least one nanoparticle, wherein degradation of the matrix releases the nanoparticle In embodiments the nanoparticle may be hollow, may be a superparamagnetic porous nanoparticle, and may contain a therapeutically effective amount of a therapeutic agent. In embodiments the matrix may comprise PVA, the therapeutic agent may inhibit the growth of a cancer and the nanoparticle may be coated with a separating agent; or a targeting agent; or a separating agent and a targeting agent.

In an alternative embodiment there is disclosed a method of selectively obstructing blood flow in a blood vessel in a patient, the method comprising the steps of: administering to the patient an embolization composition for purposively inducing an embolism in said patient blood vessel.

In an alternative embodiment there is disclosed a method for delivering a therapeutic agent to a patient, the method comprising the steps of: administering to the patient an embolisation composition that contains the therapeutic agent; and releasing the therapeutic agent.

In alternative embodiments the method further comprises introducing said embolisation composition into a blood vessel supplying blood to a target region in said patient. Such localising may use a selective cathetier and said target region may comprise a tumor.

In alternative embodiments the method further comprises the step of applying a magnetic field to position the embolisation composition.

In alternative embodiments there is disclosed a method for making an embolisation composition, the method comprising the step of embedding at least one nanoparticle in a degradable matrix to thereby form the embolisation composition.

In alternative embodiments a nanoparticle may contain a therapeutic agent.

In alternative embodiments a nanoparticle may be a hollow, superparamagnetic nanoparticle.

In alternative embodiments the matrix suppresses the release of the therapeutic agent.

In alternative embodiments the therapeutic agent may be an anticancer agent.

In alternative embodiments there is disclosed a method for inhibiting growth of a cancer, the method comprising the step of inhibiting the blood supply to the cancer with a degradable embolisation composition, the embolisation composition comprising a degradable matrix, a plurality of magnetic nanoparticles, and a therapeutically effective amount of an anticancer agent.

In alternative embodiments the nanoparticles are embedded in matrix, and the method further comprises the steps of: allowing ones of the nanoparticles to escape from the blood vessel carrying the blood supply; and releasing the anticancer agent proximate the cancer.

In alternative embodiments the nanoparticles may be embedded in the matrix and may carry the therapeutic agent, and wherein the method may further comprise the steps of: allowing ones of the nanoparticles to escape from the blood vessel carrying the blood supply; and releasing the anticancer agent from the nanoparticles.

Embodiments disclosed include magnetic particles of variable sizes, methods for making the particles and methods for controlled release of therapeutic agents using the particles. In alternative embodiments there is disclosed a porous magnetic nanoparticle; the particle may be hollow or may comprise a polymer or may be adapted for controlled release of a therapeutic agent. In an alternative embodiment there is disclosed a drug delivery system comprising a hollow, porous magnetic nanoparticle. The magnetic nanoparticle may be associated with a polymer. The system may be adapted to controlledly release a therapeutic agent and may be a chemoembolisation system.

In an alternative embodiment there is disclosed a therapeutic composition comprising a plurality of nanoparticles and a matrix. In an alternative embodiment the therapeutic composition may further comprise a pharmaceutically active therapeutic agent.

In an alternative embodiment the compositions of other embodiments are used to locally deliver a therapeutic agent. In alternative embodiments the particle may be of substantially predetermined size.

Features and advantages of the subject matter hereof will become more apparent in light of the following detailed description of selected embodiments, as illustrated in the accompanying figures. As will be realized, the subject matter disclosed and claimed is capable of modifications in various respects. Accordingly, the subject matter set out herein is to be regarded as illustrative in nature.

### BRIEF DESCRIPTION OF THE DRAWINGS

FIG. 1 shows selective catheter delivery of embolisation compositions according to an embodiment.

FIG. 2 shows the leakage of nanoparticles according to an embodiment through apertures in blood vessels.

FIG. 3 shows the magnetic localisation of nanoparticles or embolisation compositions in a subject's body according to an embodiment.

FIG. 4 shows the process of forming nanoparticles, and embolisation bodies and using them for chemoembolisation according to an embodiment.

FIG. 5 shows a synthetic scheme for an iron oxide nanoparticle according to an embodiment.

FIG. 6A is an SEM image of polystyrene (PS) nanospheres according to an embodiment.

FIG. 6B is a TEM image of polystyrene (PS) nanospheres according to FIG. 6A.

FIGS. 6C and 6D are TEM images of PS/Fe3O4 nanostructures of embodiments.

FIGS. 7A and 7B are TEM images of iron oxide nanoshells of embodiments at two different magnifications.

FIG. 8A is a photograph showing (from left to right) hollow porous iron oxide nanoshells, PVA, and two examples of embolisation compositions comprising iron oxide nanoparticles and PVA.

FIG. 8B shows isolated nanoparticles,

FIG. 8C shows uncombined PVA;

FIGS. 8D and 8E are photographs of embolisation compositions comprising iron oxide nanoparticles and PVA according to an embodiment.

FIGS. 9a, 9b and 9c are photographs at different magnifications of the iron oxide nanoshell/PVA embolisation compositions of an embodiment in media.

FIGS. 9d, 9e, and 9f are photographs showing the degradation of a single iron oxide nanoparticle/PVA embolisation composition attached to the wall of a vial at different time points (9d 1 hr, 9e 4 hr, 9f 6 hr).

FIG. 10 illustrates the breakdown of an embolisation composition according to an embodiment.

### DETAILED DESCRIPTION OF THE INVENTION

### Definitions

In this disclosure the following terms have the meanings set forth below:

In this disclosure the singular forms an “an” or “the” include plural referents unless the content clearly dictates otherwise. Thus, for example, reference to a composition containing “a compound” includes a mixture of two or more compounds and reference to a composition that contains “a” nanoparticle, includes a composition containing any greater number of nanoparticles.

In this disclosure term “or” is generally employed in its sense including “and/or” unless the content clearly dictates otherwise.

In this disclosure the term “magnetic” includes magnetic properties of superparamagnetism, ferrimagnetism and ferromagnetism and reference to magnetic particles indicates particles having such properties. In embodiments, the nanoparticles disclosed herein may comprise a superparamagnetic form of iron oxide. Superparamagnetic iron oxide is one of the highly magnetic forms of iron oxide (which may include but are not limited to magnetite, non-stoichiometric magnetite, gamma-ferric oxide) that may have a magnetic moment of greater than about 30 EMU/gm Fe at 0.5 Tesla and about 300 K. In such a superparamagnetic material, when magnetic moment is measured over a range of field strengths, it may show magnetic saturation at high fields and may lack magnetic resonance when the field is removed.

In this disclosure a “matrix” means a polymer or other composition which may be mixed with nanoparticles or conjugated thereto and may constitute a gel, or a solid, or may otherwise aggregate the nanoparticles. In embodiments a matrix may be adapted to break down or degrade or controllably release nanoparticles and/or therapeutic agents. A matrix may comprise a range of polymers or suitable molecules and these may be biocompatible. In embodiments a matrix may be or may comprise one or more of poly[3-O-(4′-vinylbenzyl)-D-glucose] (PVG), polyethylene glycol (PEG), polyvinyl alcohol PVA), gelfoam, gelatine sponge, silica, carbon, dextran, polylactic acid, polyglycolic acid, poly(N-isopropylacrylamide) (PNIPAM), hydroxyapatite, layered double hydroxides, and alginates. A range of equivalents and variants on the foregoing possible matrix chemicals will be readily apparent to those skilled in the art and will be selected from to satisfy particular requirements. It will be appreciated that the matrix composition may be adjusted to control the rate of matrix degradation and the consequent rate of release of any nanoparticles or therapeutic agents that have been combined therewith. In embodiments where the matrix comprises PVA, in some embodiments breakdown properties of the matrix may be adjusted by selecting PVAs of different molecular weights, or alternatively PVA's having differing hydrolysis rates may be selected to determine desired breakdown and release properties. In embodiments PVA 98 and PVA 80 have been found to be suitable for some application but it will be understood that a range of alternative PVAs or other suitable components may be used to satisfy particular requirements. In alternative embodiments the molecular weight of PVA used may be between about 1,000 and about 100,000 or between 4,000 and about 60,000, about 9,000 and about 50,000, or may be between about 1,000 and 5,000, about 5,000 and 10,000, about 10,000 and 15,000, about 15,000 and 20,000, about 20,000 and 25,000, about 25,000 and 30,000, about 30,000 and about 35,000, about 35,000 and 40,000, about 40,000 and 45,000, about 45,000 and about 50,000, about 50,000 and about 55,000, about 55,000 and about 60,000, about 60,000 and about 65,000, about 65,000 and about 70,000, about 70,000 and about 75,000, about 75,000 and about 80,000, about 80,000 and about 85,000, about 85,000 and about 90,000, about 90,000 and about 95,000, about 95,000 and about 100,000 or greater. In alternative embodiments PVA may have a hydrolysation level (also referred to as a hydrolysed rate) of between about 0% and about 20%, between about 20% and about 30%, between about 30% and about 40%, between about 40% and about 50%, between about 50% and about 60%, between about 60% and about 70%, between about 70% and about 80%, between about 80% and about 85%, between about 85% and about 90%, between about 90% and about 95%, between about 95% and about 100% or may be about 80%, 81%, 82%, 83%, 84%, 85%, 86%, 87%, 88%, 89%, 90%, 91%, 92%, 93%, 94%, 95%, 96%, 97%, 98%, 99%, or about 100%.

The composition of PVA is described with respect to its “hydrolysed rate” or “hydrolysation level”. PVA (Polyvinyl alcohol) may be prepared by hydrolysing Polyvinyl acetate (PVAc), in this hydrolysing reaction, the —OOCCH3 group of PVAc is substituted with an —OH group. The “hydrolysed rate” or “hydrolysation level” refers to the substituted percentage of —OH groups in the PVA.

It will be understood that in embodiments matrix materials may be degradeable or biodegradeable, by which is meant that the matrix may break down or become less viscous or lose its integrity when exposed to a suitable environment. Suitable environments may include but are not limited blood, plasma, intercellular spaces, body fluids, aqueous environments and the like. It will be understood that breakdown or degradation of matrix may comprise chemical changes to the matrix, but may also merely reflect or also reflect the dilution of the matrix materials or the breakdown of non covalent bonds in the matrix. Thus in embodiments, the degradation of the matrix may reflect physical changes in the matrix, but may not require chemical changes in the nature thereof. In alternative embodiments the degradation of the matrix may be promoted by enzymes or other chemicals present in the environment to which the matrix is exposed. It will be further understood that matrix may comprise components selected to allow degradation of the matrix in a first environment, such as blood, and other components selected to degrade in other environments such as intercellular spaces. Thus, in embodiments the matrix may degrade in phases, thus delaying release of any therapeutic agent until nanoparticles containing the agent have dispersed or moved through a blood vessel wall into the surrounding tissue.

In this disclosure, a “nanoparticle” or a “particle” (which terms are used interchangeably) means a particle which may be hollow, may be perforated by one or more openings or nanopores, may be porous, and may be comprised of a magnetic material, which may be a superparamagnetic material, and may comprise a metal oxide which may be an iron oxide. In embodiments a nanoparticle may be coated, may be conjugated and may be associated with or encapsulated in a matrix. The term nanoshell is also used herein to refer to a hollow nanoparticle. While methods for making nanoparticles are disclosed herein, it will be understood that these methods are presented by way of example only, and that in particular alternative embodiments a wide range of nanoparticles made by any conventional methods may be suitable for use in the embolisation compositions disclosed herein.

In embodiments, individual nanoparticles, whether coated or uncoated, may have an overall size or diameter of about 1 nm to about 500 nm, about 1 to 50 nm, about 1 to 20 nm, about 5 to 15 nm, about 50 to 100 nm, about 50 to 100 nm, about 50 to 150 nm, about 50 to 200 nm, about 50 to 250 nm, about 50 to 300 nm, about 50 to 350 nm, about 50 to 400 nm, about, about 1 to 100 nm, about 1 to 150 nm, about 1 to 200 nm, about 1 to 250 nm, about 1 to 300 nm, about 1 to 350 nm, about 1 to 400 nm, about 10 to 50 nm, about 10 to 100 nm, about 10 to 150 nm, about 10 to 200 nm, about 10 to 250 nm, about 10 to 300 nm, about 10 to 350 nm, about 10 to 400 nm, about 100 to 200 nm, about 200 to 300 nm, about 300 to 400 nm, about 400 to 500 nm or may be greater than about 1, 5, 10, 20, 50, 100, 200, 300, 400, 500 nm or less than about 500, 400, 300, 200, 100, 150, 100, 50, 40, 30, 20, 10, or 5 nm or the overall diameter may fall in the same ranges. While a wide range of sizes is possible, in particular embodiments the nanoparticle may have a diameter in a range selected from the group comprising: between about 1 nm and about 500 nm, between about 1 nm and about 400 nm, between about 1 nm and about 300 nm, or between about 1 nm and 200 nm, or between about 1 nm and 100 nm or between about 50 and 300 nm. Such dimensions may include or exclude any coating or conjugation.

The nanoparticles disclosed may be hollow, may be porous, and may comprise openings, or pores which may also be referred to as nanopores, suitable to allow release from the nanoparticles of a therapeutic agent contained in the nanoparticle. In embodiments a matrix may be associated with or comprised in or may coat the nanoparticles and may permit the controlled release of a therapeutic agent contained or held in the nanoparticles. In embodiments a nanoparticle may be modified to affect its hydrophobicity or hydrophilicity to modify the release of the desired therapeutic agent.

In this disclosure the statement that a nanoparticle is “functionalized” or “coated” means that the nanoparticle (with or without a coating) has been treated to bear functional groups. In embodiments such functional groups may be or may include amine, ammonium, alkylamine, dialkylamine, amide, hydroxyl, ether, carboxyl, ester, thiol, thioether, alkene, alkyne NH₂, N⁺H₃, NHR, NR₂, C(O)NHR, OH, OR, COOH, COOR, SH, SR, C═CH₂, C═CHR, C═CR₂, C≡CH, C≡CR, aromatics (where R=includes straight and branched alkyl chains, ring structures and combinations of the foregoing). In embodiments, the nanoparticles may be coated with or associated a wide range of materials including but not limited to reactive, inert, amphiphilic, polar and non polar, biologically or chemically active materials, and in embodiments possible coatings may consist of or comprise silica, dextran, polyethylene glycol, polylactic acid, polyglycolic acid, alginate, poly(N-isopropylacrylamide) (PNIPAM), hydroxyapatite, layered double hydroxide and alginate. Coatings may be chosen to modify the interaction of the nanoparticles with their associated therapeutic agents, or with the matrix, or both, all in ways that will be readily understood by those skilled in the art. In further embodiments the nanoparticles may further comprise or may be associated or conjugated with a contrast agent suitable to enhance particular forms of detection, for example for use in MRI. In embodiments, blood residence time of nanoparticles may be prolonged, by chemically binding a stabilizing agent to the magnetic particle surface.

In this disclosure the term “conjugate” or “conjugation” or the like of nanoparticles means linking of the nanoparticles to chemicals or materials and the terms “bioconjugate” or “bioconjugation” and like terms indicates conjugating the nanoparticles to biomolecules. In embodiments, conjugation of a nanoparticle may be to any desired chemicals, molecules, complexes, structures, biomolecules, bioactive chemicals and the like. In embodiments the nanoparticles may be conjugated with a range of pharmaceuticals, chemicals or materials, for particular purposes and in particular embodiments conjugated pharmaceuticals may comprise anti-cancer drugs. Suitable biomolecules may include but are not limited to proteins, nucleic acids, DNA, RNA, carbohydrates, lipids, antibodies, lectins, streptavidin, proteins, enzymes, hormones, vitamins, ligands, receptors, pharmaceuticals, Doxorubicin, Taxol, cisplatin, Traditional Chinese Medicines, and all manner of biological or biologically active molecules. Those skilled in the art will readily select suitable conjugates, including bioconjugates and suitable methods of conjugation to suit particular purposes. Generally conjugation may be accomplished by means of covalent linkages but in embodiments it may be carried out using other forms of linkage. In embodiments the conjugates or the coating of the nanoparticles may be usable to target the nanoparticles to specific cell types and locations or to modify their properties for specific purposes. Those skilled in the art will readily understand how to make suitable modifications to achieve these purposes.

In embodiments nanoparticles may be coated or conjugated to targeting agents and in this disclosure the term “targeting agent” (also referred to a localising agent) means a reagent or property whose function or purpose is to promote localisation of the nanoparticle or embolisation composition in proximity to a desired target region in a patient. By way of example and not limitation, a target region may be or comprise a selected tissue, organ or cell type and may comprise a cancer and may be a tumor. Thus in embodiments targeting agents may comprise, for example, suitable antibodies, lectins, proteins, polypeptides, carbohydrates or lipids or combinations of the foregoing.

In embodiments a nanoparticle may comprise a metal oxide. In embodiments the oxide may be of a bivalent or trivalent metal, may be an oxide of a lanthanide or rare earth metal, or may be an oxide of Iron, Cobalt, Titanium, Manganese, Magnesium, Nickel, Copper, Zinc, Vanadium, Berylium, Barium, Strontium, Gold, Palladium, or Platinum, or may be or may comprise Aluminium, Ytterbium, Yttrium, Manganese or Chromium, or alternative metals or mixtures of or comprising one or more of the foregoing. Those skilled in the art will readily select between such metals and combinations of metals for particular purposes taking into account their physical properties, cost, toxicity and the like. In particular embodiments the nanoparticles may be iron oxide nanoparticles.

In embodiments the nanoparticles may be made or rendered water soluble using various techniques, such as surface modification. Those skilled in the art will readily understand and implement a range of modifications to nanoparticles such as the additional encapsulation of individual nanoparticles with molecules, or polymer, or biodegradable polymers such as polyvinyl alchohol (PVA), polylacetic acid (PLA), polyglycolic acid (PGA), PLGA (PLA-co-PGA), poly(N-isopropylacrylamide) (PNIPAM), dextran, hydroxyapatite, layered double hydroxide and alginate and such as functionalization of the nanoparticle with desirable functional groups.

In this disclosure the term “embolisation composition”, “embolisation body” or “embolisation system” means an aggregation, agglomeration, condensation or other composition comprising at least one nanoparticle and a matrix. In embodiments an embolisation composition will generally contain a plurality of nanoparticles and a matrix, together forming a body of chosen dimensions. In particular embodiments an embolisation composition may be substantially spherical or may be substantially cubic or substantially elongate or may have any desired shape. Embolisation compositions may be shaped by extrusion, controlled formation or polymerisation, by cutting or any other shaping means. Embolisation compositions may have any desired dimension, and in particular embodiments their overall diameter may be between about 0 mm and 1 mm, between about 1 mm and 2 mm, between about 2 mm and 3 mm, between about 3 mm and 4 mm between about 4 mm and 5 mm or bigger. In alternative embodiments the diameter of an embolisation composition may be between about 0.5 mm and 4 mm, between about 1 mm and 3 mm, or between about 1 mm and 2 mm, or an embolisation composition may be about 1.5 mm in diameter. In embodiments embolisation compositions may be coated and may comprise targeting agents, separating agents, excipients, therapeutic agents, buffer, diluents and the like. In embodiments, embolisation bodies may be chemoembolisation bodies and may comprise desired therapeutic agents for release proximate a target region, which may comprise a tumor.

In this disclosure the term “separating agent” or “stabilising agent” means a chemical or reagent which suppresses, reduces or modulates clumping of nanoparticles or of embolisation compositions or which enhances the stability or blood residence time of the nanoparticles or embolisation compositions, or which otherwise modifies the physicochemical properties of the nanoparticles or embolisation compositions as may be desired by a user. Stabilising agents which may be used in this way may include carbohydrates such as oligo- and polysaccharides, as well as polyamino acids, oligo- and polynucleotides and polyalkylene oxides (including poloxamers and poloxamines) and other materials. In embodiments nanoparticles or embolisation compositions my have chemically or physically bonded to the surface thereof a hydrophilic blood-lifetime-prolonging polymer which may, by way of example and not of limitation, be a functionalized polyalkyleneoxide such as methoxy-PEG-phosphate, and may be a terminally functionalized linear polymer.

In this disclosure the terms “obstructing”, “occluding”, “blocking”, “embolising” and like terms, where used herein with reference to blood vessels and the flow of blood therethrough, are to be understood in their broadest sense and include both complete and partial occlusion of the vessel or blood flow therethrough. It will be understood that embolisation compositions may be used to obstruct, occlude, block or embolise blood vessels.

In this disclosure the term “chemoembolisation” means obstructing, blocking, occluding or embolising a blood supply to a target region, in combination with the local delivery of suitable therapeutic agents proximate the target region. Such therapeutic agents may be carried by an embolisation composition.

In this disclosure the term “scaffold” or “core” means a structure upon or around which a nanoparticle may be formed, deposited or otherwise created. In particular embodiments a nanoparticle may be formed by creating a scaffold of the desired size, depositing the body of a nanoparticle thereon, allowing the deposition of the nanoparticle material, which may be a metal oxide and may be an iron oxide, to proceed until the deposited material forms a nanoparticle of a desired thickness, diameter and properties, and then degrading the scaffold to leave a hollow nanoparticle.

In this disclosure the term “hollow” is to be understood broadly and may comprise any form of cavity or recess, void, or depression, or other structural feature comprised in a nanoparticle, that is capable of containing, holding, or delimiting a selected substance, which may be a therapeutic agent. Thus a hollow nanoparticle may have a single internal cavity, or may comprise one or more cavities or tubes or openings and one or more of such cavities, tubes or openings may communicate with the external environment of the particle so that a substance contained in the particle can thereby be communicated to the medium.

In this disclosure the term “porous” where used with reference to a nanoparticle, means that the nanoparticle is structured to permit the escape or release of a given agent contained within the nanoparticle. In embodiments such an agent may be a therapeutic agent.

In embodiments the nanoparticles disclosed may be delivered or administered to an organism or tissue or cells or to a patient using a suitable medical device, and may be injected or may be applied orally, topically, transdermally, subcutaneously, intraperitoneally, intraocularly, intracranially, intracerebroventricularly, intracerebralyl, intravaginally, intrauterinely, orally, nasally, rectally or parenterally (e.g., intravenously, intraspinally, subcutaneously or intramuscularly) subcutaneously, intravascularly, by catheter, or by any other conventional or suitable method, the full range of which will be readily understood and implemented by those skilled in the art. In alternative embodiments the nanoparticles may be delivered systemically, or locally, or their delivery or behaviour may be modified using a range of conventional methods that will be readily understood and implemented by those skilled in the art. In embodiments the delivery of the nanoparticles to a desired target location may be directed, driven, assisted, or contributed to by internally or externally applied magnetic fields.

In this disclosure the terms “cancer” or “malignancy” or “tumour” mean and include all forms of cancer, including but not limited to cancers of the bladder, brain, breast, liver, cervical, colorectal, uterine, esophagus, Hodgkin lymphoma, kidney, larynx, leukaemia, lip, lung, multiple myeloma, non-Hodgkin lymphoma, oral cavity, ovary, pancreas, prostate, skin, stomach, testis, and thyroid. The terms also include both developed cancers and all forms of precancerous cells that may have or be predicted to have a predisposition to develop into cancerour cells.

In this disclosure the term “therapeutic agent” means any active pharmaceutical ingredient or any agent having actual or potential therapeutic effects on a subject. In embodiments any therapeutic agents may be combined or associated with suitable excipients. In embodiments the therapeutic agents may be anticancer agents which may be agents suitable for inhibiting the growth of a cancer or of cancer cells. By “inhibiting” is meant that the agent may prevent or slow or otherwise modify the division or survival or metabolism or chemical processes or maturation or other aspects of the cancer cells.

In this disclosure the terms “deliver” or “introduce” a therapeutic agent mean applying, injecting, dosing, administering or in any way delivering or introducing the therapeutic agent to any part of the body of a patient or subject.

In this disclosure the term “controlled release” means that a therapeutic agent is released from a nanoparticle or from a population of nanoparticles over a controlled or predetermined period or following a predetermined release profile, or that nanoparticles are released from a polymer or other matrix over a predetermined period or following a predetermined profile. In embodiments the controlled release profile or time course of release of a therapeutic agent or of a nanoparticle or a population of nanoparticles may be modified by changing the ratio of particles to matrix, polymer or conjugate in a preparation of nanoparticles, by changing the matrix, polymer or conjugate with which the particles are associated, by changing the pore size of the nanoparticles or by such other forms of manipulation and modification as are known to those skilled in the art.

Compositions according to embodiments may be formulated in any conventional pharmaceutical form, including but not limited to suspensions, dispersions, powders, and the like, and may contain aqueous vehicles (such as water for injections) and may contain further ingredients to adjust osmolality, pH, viscosity, and stability. In embodiments the compositions may be in suspension form with the suspension being isotonic and isohydric with blood. For example, an isotonic suspension may be prepared by the addition of suitable salts, such as sodium chloride or potassium chloride, low-molecular weight sugars such as glucose (dextrose), lactose, maltose, or mannitol or other suitable chemicals or combinations of chemicals. Isohydricity may also be achieved by the addition of suitable acids or bases or by the use of buffers such as phosphate, citrate, acetate, borate, tartrate, and gluconate. The chemical stability of embodiments may also be modified by the addition of antioxidants, for example ascorbic acid or sodium pyrosulphite and chelating agents, for example citric acid, sodium EDTA and sodium calcium EDTA. In embodiments, excipients and stabilisers may also be added to improve the physical stability of preparations.

In this disclosure the term “carrier” or “excipient” means and includes all suitable compositions which will be acceptable in the sense of being compatible with the other ingredients of the composition and are not significantly deleterious to the recipient. The carrier or excipient can be a solid or a liquid, or both, and may be formulated with the compound of the invention as a unit-dose composition, for example, an embolisation composition or group thereof, which can contain from 0.05% to 95% by weight of the active compound. Such carriers or excipients include inert fillers or diluents, binders, lubricants, disintegrating agents, solution retardants, resorption accelerators, absorption agents, and coloring agents. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes, and the like lubricants include sodium oleate, sodium stearate, magnesium stearate, sodium benzoate, sodium acetate, sodium chloride, and the like. Disintegrators include starch, methyl cellulose, agar, bentonite, xanthan gum, and the like. Possible components of an excipient of particular embodiments may be or may comprise mannitol, pregelatinized starch, magnesium stearate, sodium saccharine, talcum, cellulose ether derivatives, gelatin, sucrose, citrate, propyl gallate, lactose, white soft sugar, sodium chloride, glucose, urea, starch, calcium carbonate, kaolin, crystalline cellulose, silicic acid, calcium silicate, potassium phosphate, cacao butter, hardened vegetable oil, kaolin, and others, all of which will be readily apparent to those skilled in the art.

Embodiments of the subject matter claimed and disclosed are described with general reference to FIG. 1 through 10. The labelled and their respective reference numerals used in the drawings include the following: emoblisation composition 100, catheter 102, blood vessel 104, tumour 106, smaller blood vessels 104 (which may be arteries or arterioles) and narrower blood vessels 108 (which may be areterioles or capillaries), blood vessel wall 109, lumen 122 of blood vessels, apertures in blood vessel wall 110, nanoparticles 120, matrix 122, therapeutic agent 130, magnetic field 140, magnet or other or equivalent device for generating a magnetic field 142, body or subject/patient 150, cells 152 in blood vessel wall; polystyrene cores or scaffolds 200, nanoshell walls 210, cavity within nanoshell 230, efferent blood vessels 105 may be capillaries, veinules or veins.

### First Embodiment

In a first embodiment there are disclosed embolisation compositions, and uses of the embolisation compositions to selectively obstruct blood vessels in a patient, or to deliver a therapeutic agent to a desired target region in a patient.

An embolization composition may comprise a matrix and at least one nanoparticle. The matrix may be biodegradable and may encapsulate or restrain the at least one nanoparticle. In embodiments the degradation of the matrix may release the nanoparticle. In embodiments the compositions may comprise a plurality of nanoparticles. In alternative embodiments, the nanoparticle may be hollow, may be superparamagnetic, and may be porous and may contain a therapeutic agent which may be an anticancer agent. Embolisation compositions and nanoparticles may contain a therapeutically effective amount of a therapeutic agent. The matrix may comprise PVA, the therapeutic agent may be suitable to inhibit the growth of a cancer, and the nanoparticle may be coated with a separating agent; or a targeting agent; or a separating agent and a targeting agent.

Referring particularly to FIGS. 4, 5 and 10, nanoparticles 120 may be porous and may comprise or may consist of a metal compound which may be a metal oxide and may be an iron oxide. The nanoparticles 120 may be embedded or encapsulated in a matrix 122, and matrix and nanoparticles together may form an embolisation composition 100. The matrix 122 may be degradable and may be biodegradable, and may comprise one or more biocompatible polymers, such as polyvinyl alcohol (PVA) or other suitable alternatives, to form an embolisation composition 100 that may be several millimetres in diameter, and may be about 1.5 mm in diameter. It will be understood that these dimensions are by way of example only and may be adjusted for particular applications. A range of alternative sizes and matrix compositions are set forth elsewhere within this disclosure.

In variants of the embodiments a nanoparticle 120 may contain a therapeutic agent 130 which may be an anticancer agent. The nanoparticles 120 may be between about 100-200 nm in diameter but may be formed into any other suitable size, and a range of examples of alternative possible sizes are set out elsewhere within this disclosure. The particles may have an empty core (i.e. they may be nanoshells) or may have multiple pores (also referred to as nanopores) therein. The nanoparticles 120 may be filled or associated with a therapeutic agent 130 or agents, which may comprise an anti-cancer agent, or a combination of anti-cancer agents. The matrix 122 may be biodegradable or degradable, and may comprise one or more biocompatible polymers, such as polyvinyl alcohol (PVA).

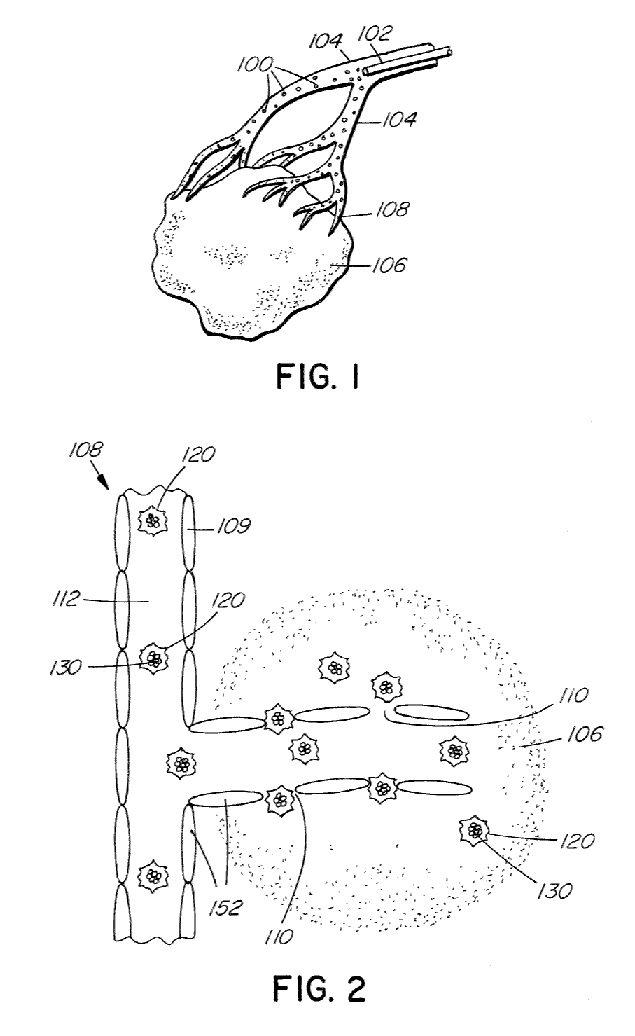

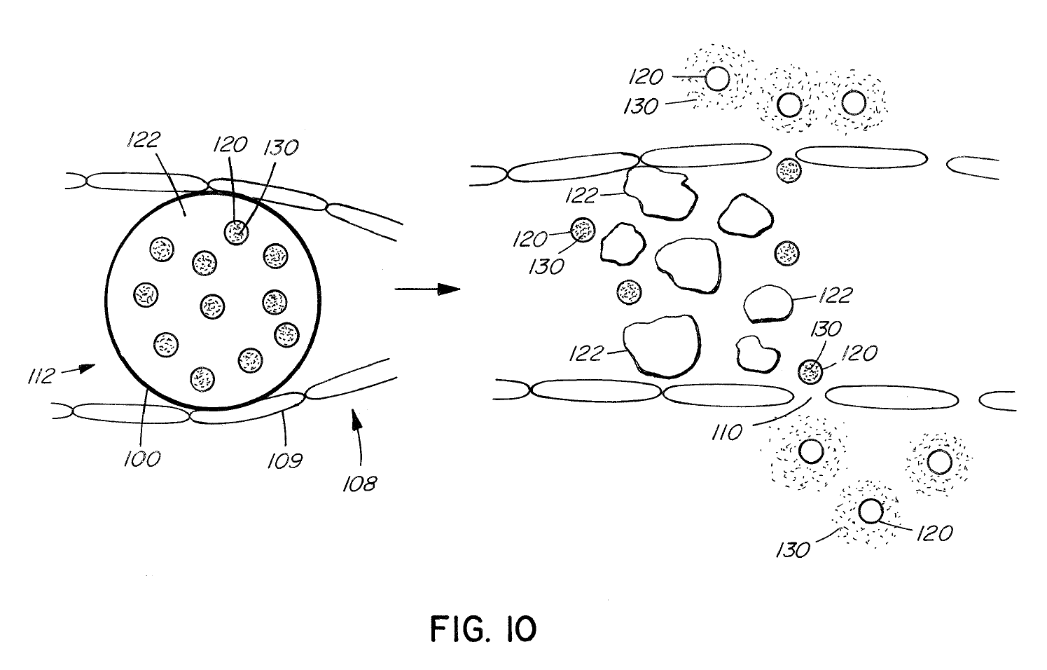

In a further variant of the embodiment generally described with particular reference to FIGS. 1, 4 and 10, there is disclosed a method of selectively obstructing blood flow in a blood vessel 104, or 108 in a patient, the method comprising the steps of administering to the patient an embolization composition 100 according to the embodiment for purposively inducing an embolism in the patient blood vessel 104, 108. The embolisation compositions may be introduced into the blood vessel using a catheter 102. Thus in operation an embolisation composition 100 or a plurality of such embolisation compositions, may be introduced into a blood vessel 104 which leads to a target region, which may be or may comprise a tumour 106. It will be seen that as embolisation compositions 100 are carried into smaller blood vessels 108 they will eventually reach a point where the diameter of the embolisation composition 100 is larger than the internal diameter of the smaller blood vessel 108 (which may be an arteriole or capillary) and will then be unable to move further, being lodged between the vessel walls 109 in the lumen 112 of the vessel. The lodging of the embolisation composition 100 may result in a partial or complete occlusion or obstruction of the blood flow through the blood vessel 104, 108 as illustrated in FIG. 10. This method of introducing the chemoembolisation compositions 100 to a desired location may minimize side effects on other tissues. Once an embolisation composition 100 has lodged in a blood vessel 104 or 108, then as seen particularly in FIG. 10, FIG. 4, and FIG. 2, the matrix 122 of the embolisation composition 100 may degrade, and the nanoparticles 120 encapsulated or enmeshed in the matrix 122 may thus be released, as may any therapeutic agent 130 associated therewith.

In variants of the embodiments the nanoparticles 120 may contain a therapeutic agent shown schematically as 130, and the degradation of the matrix 122 may release the therapeutic agent 130. It will be understood that by changing the composition of the matrix 122 or any outer layer that may be applied to encapsulate the embolisation composition 100, or the properties of the nanoparticles themselves, the nanoparticles and any therapeutic agent 130 may be released in a controlled manner, for instance release may be delayed or extended over a desired time period. If the embolisation composition 100 has been localised in a blood vessel 108, which may be a small artery or arteriole feeding a tumour 106, then the therapeutic agent 130 will be locally released. It will be understood that in embodiments where matrix material prevents or limits the release of a therapeutic agent from the nanoparticles 120, then it may be desirable to provide matrix with degradation properties that allow the nanoparticles 120 to dissociate so as to pass through the capillary wall, while still retaining sufficient associated matrix material to prevent the immediate release of the therapeutic agent. Thus for certain applications it may be desirable that the matrix breaks down at such a rate that the nanoparticles are able to dissociate in a blood vessel 108 but that any pores therein take longer to become unobstructed so that the therapeutic agent is released in the surrounding tissue rather than in the lumen of the blood vessel 108.

In further variants of the embodiment, there is disclosed a method for delivering a therapeutic agent 130 to a patient or to a target region within a patient, the method comprising the step of administering to the patient a nanoparticle 120 which may contain the therapeutic agent 130. The nanoparticle 120 may be comprised in an embolisation composition 100. Delivering the therapeutic agent 130 to a patient may comprise the steps of: administering to the patient an embolisation composition 100 according to an embodiment, wherein the embolisation composition 100 contains the therapeutic agent 130; and releasing the therapeutic agent. The method may further comprise introducing the embolisation composition 100 into a blood vessel 104, 108 supplying blood to a target region in said patient. The target region may be or may comprise a tumor. Delivering the therapeutic agent may comprise degrading the matrix 122 of the embolisation composition 100 to release the therapeutic agent 130.

As illustrated in FIG. 2 and FIG. 10, in some cases the vasculature of a tumour 106 or target region may be leaky and thus nanoparticles 120 may escape from the blood vessel 108 or 104 into the interstitial tissues of the tumour 106 through openings 110 between the cells 152 in the walls of the blood vessel which may be a capillary. The nanoparticles 120 may thus deliver the therapeutic agent 130 at that location. In other regions of the subject's or patient's body 150 where the vasculature is not leaky, the particles will not generally escape from the blood vessels.

Nanoparticles may accumulate in and around the tumour and as the matrix components or other sealing agents degrade, anti-cancer agents or other therapeutic agents can then be slowly released in the tumor region. Additionally, nanoparticles may have the ability to accumulate in tumor cells resulting in the increased intracellular concentration of drugs. In embodiments this may be facilitated or promoted by coating the nanoparticles with suitable targeting agents, optionally including suitable chemicals, amine groups or suitably reactive antibodies. Thus the delivery of the therapeutic agent 130 may be locallised by positioning the embolisation compositions 100 in or near a desired target region.

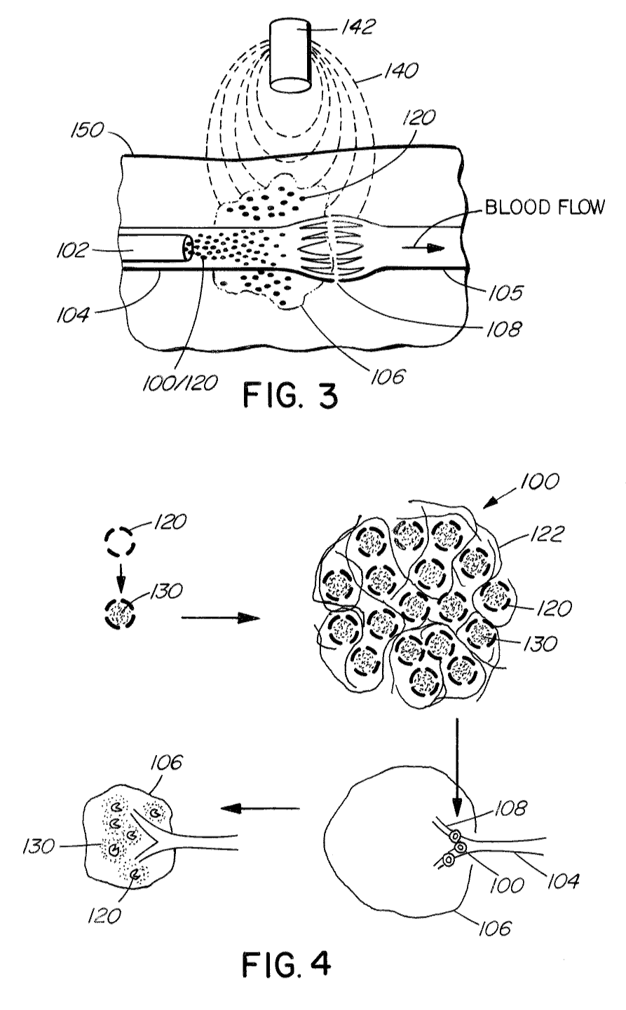

As illustrated in FIG. 3, in alternative embodiments the nanoparticles 120 and the embolisation compositions 100 may be localised within the body of the subject by the use of an applied magnetic field 140 generated by a magnet or other suitable device 142 focussed on the target area of the body which may comprise a tumour 106 or other target tissue. Similarly in embodiments the distribution of the nanoparticles 120 and embolisation compositions 100 may be visualised by magnetic resonance imaging. It will be seen in FIG. 3 that embolisation bodies 100 or nanoparticles 120 may be introduced into an arterial or other blood vessel 104 using a catheter 102. The embolisation bodies or nanoparticles will be carried in the bloodstream to narrower blood vessels 108 which may be capillaries or arterioles, these then lead to efferent blood vessels or veins 105. An applied magnetic field 142 may both prevent movement of the nanoparticles 120 and/or embolisation compositions 100 out of the region subject to the magnetic field 140, and the embolisation bodies and nanoparticles may also be actively spread into the surrounding tissues under the influence of the magnetic field. Where embolisation compositions are introduced then they may lodge in the smaller blood vessels 108 and may break down there, liberating nanoparticles which may then escape from the blood vessel 108 under the influence of the applied magnetic field 140. It will be understood that if the nanoparticles 120 are loaded with a therapeutic agent 130 then this agent may be carried into the surrounding tissue with the nanoparticles 120.

It will be appreciated that the nanoparticles 120 may be composed so that an associated therapeutic agent 130 is not liberated immediately on degradation of matrix 122, but is further delayed or controlled. For example, individual nanoparticles 120 may be associated with a slower degrading matrix composition than the bulk of matrix 122, or may be chemically modified to delay or control the release or dissociation of the therapeutic agent 130 from the nanoparticles 120.

While the foregoing explanation of the embodiment contemplates the use of the nanoparticles and embolisation compositions to treat a tumor, it will be readily understood by those skilled in the art that the embodiments and their variants may equally be used to treat any other tissue or structure supplied by suitable blood vessels. Those skilled in the art will readily make such changes to the size and composition of the nanoparticles and therapeutic agents as may be desirable for particular applications and will readily select suitable therapeutic agents and methods and locations for administration to suit a full range of possible applications.

### Second Embodiment

In a second series of embodiments there is disclosed a method for making an embolisation composition or compositions 100 comprising nanoparticles, the method comprising the step of embedding at least one nanoparticle 120 in a degradable matrix 122. In alternative embodiments the nanoparticle 120 may contain a therapeutic agent 130 and degradation of the matrix 122 may release the nanoparticle 120 and the nanoparticle 120 may release the therapeutic agent 130 and may controllably release the therapeutic agent 130. In variant forms of the embodiment the nanoparticle 120 may be a hollow, superparamagnetic nanoparticle. In variants of the embodiment the matrix may suppresses the release of a therapeutic agent from the nanoparticles. In variants of the embodiment therapeutic agent may be an anti-cancer agent. In variants of the embodiment the nanoparticle may be coated with a separating agent; or a targeting agent; or a separating agent and a targeting agent.

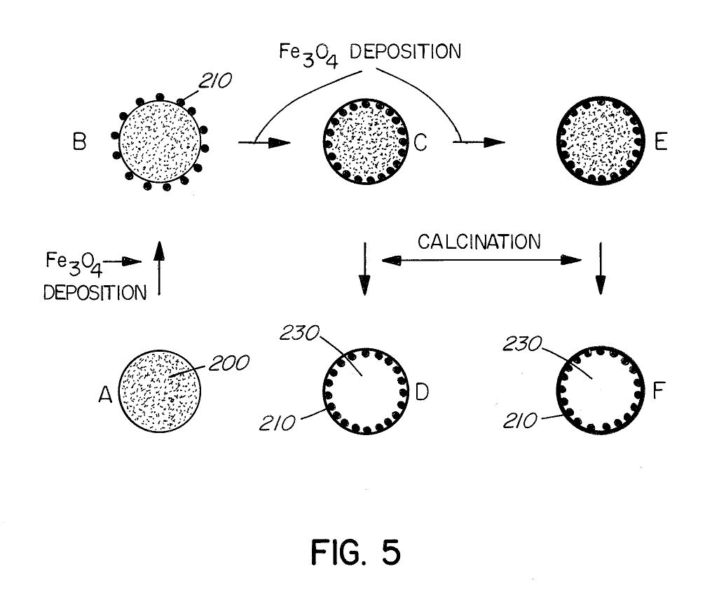

There is also disclosed a method for making a hollow magnetic nanoparticle for use in the embolisation compositions, the method comprising the step of depositing a magnetic material on a biodegradable core. The nanoparticles of the embodiment may be porous. The method is generally illustrated with particular reference to FIG. 5. As shown in FIGS. 5A and 5B, a degradable core 200 of suitable size is provided and may be a polystyrene (PS) core, and a nanoparticle shell 210 is deposited thereon and may be Fe₃O₄. Thus the structure illustrated in FIG. 5B represents a PS/Fe₃O₄ composite nanoparticle. The deposition process may be continued for a desired period, thus shorter periods of deposition may lead to a relatively thin nanoparticle shell, as shown in FIG. 5C, while if deposition is allowed to proceed further then a thicker walled nanoparticle 5E may be produced. When the core or scaffold is degraded by calcination or other suitable treatment then a thinner or thicker walled nanoshell/nanoparticle having a wall 210 and an internal cavity 230 will be produced as seen in FIGS. 5 D and F respectively.

In embodiments the scaffold or core 200 may be negatively charged carboxyl-stabilized polystyrene (PS) latex with an average diameter of 200 nm. Then iron oxide may be deposited on the surface of the scaffold particles by mixing the scaffold particles with FeCl₃ in ethylene glycol aqueous solution in the presence of hexamethylene tetramine. Further details of one exemplary method of carrying out this step are set out in the Examples.

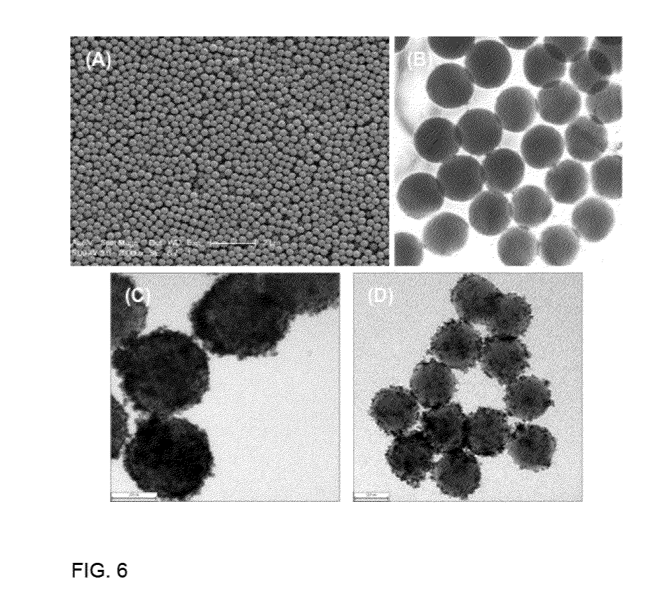

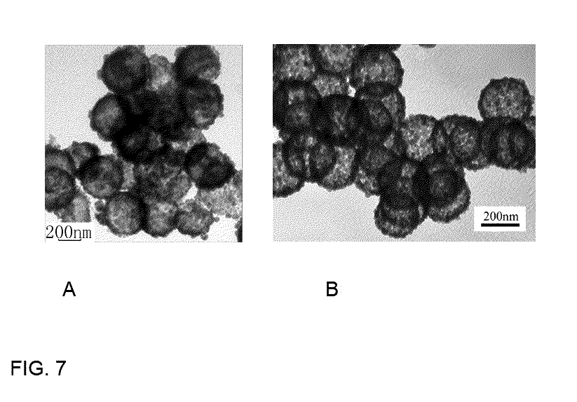

The precipitated coated core/nanoparticle combinations, which may be PS/Fe₃O₄ composites, may be washed and dried as illustrated in FIGS. 6C and 6D. The core may then be removed by any suitable means, where the core is a PS scaffold then it may be removed by calcination at suitable temperature and under suitable conditions. In embodiments oxygen may be excluded and calcination may be carried out at between about 400° C. and about 500° C. Details of one exemplary procedure for removing the cores from formed nanoparticles are presented in the Examples. The nanoparticles may be mesoporous iron oxide nanoshells, examples of which are shown in FIG. 7.

In embodiments and by continuous tuning the average size of the core or scaffold may be controllable to be from 100-200 nm in the first synthetic step, although it will be understood that by suitable adjustments to the synthetic process the size of the core particles may be adjusted to fall within a desired range of diameters, which may be from about 10 nm to about 50 nm, about 50 nm to about 100 nm, about 100 nm to about 150 nm, about 150 nm to about 200 nm, about 200 nm to about 250 nm, about 250 nm to about 300 nm, or more than about 300 nm, or less than about 10 nm or between about 100 nm and about 300 nm

In an embodiment the as-synthesized nanoparticles (nanoshells), formed by deposition on a suitable scaffold may be nearly monodispersed and may be iron oxide nanoshells. Their average sizes may be controllable by suitable adjustments to the conditions for deposition of the nanoparticle material. In particular embodiments the diameter of the nanoshells may be between about 150 and 200 nm. Examples of such nanoparticles characterized by TEM are shown in FIG. 7. It will be understood that the diameters of the nanoparticles may be adjusted to fall within many different ranges, which will be selected amongst by those skilled in the art. For example, in particular embodiments the nanoshells forming the nanoparticles may have diameters between about 10 and 50 nm, about 50 nm and 100 nm, about 100 nm and about 150 nm, about 150 nm and about 200 nm, about 200 nm and about 250 nm, about 250 nm and about 300 nm, between about 300 nm and about 400 nm, or more than about 400 nm. In particular embodiments the core materials may be degraded in a range of ways readily understood by those skilled in the art, and the size of the core and the thickness and properties of the metal, or metal oxide, nanoparticle, will all be readily adjusted by suitable changes to reaction conditions, all in ways readily understood by those skilled in the art.

In one variant of the method, iron oxide nanoparticles with a diameter of about 200 nm may be prepared by using a polystyrene acrylate templating method. One suitable core or scaffold may be charged carboxyl-stabilized polystyrene (PS) latex with an average particle diameter of about 150 nm to 200 nm. It will be understood that alternative dimensions and chemicals are possible and will be readily selected and implemented by those skilled in the art.

The preparation of a template of this general type is shown in FIGS. 6A and 6B. FIG. 6A is a scanning electron microscope (SEM) image of PS nanospheres and FIG. 6B is a transmission electron microscope (TEM) image of PS nanospheres made according to an embodiment For a PS template or core, styrene and acrylic acid monomer may be copolymerised in an emulsion using sodium dodecyl sulfate as the surfactant and ammonium persulfate as the initiator. The ratio of styrene and acrylic acid may be about 100:2 (w/w) and the reaction may be carried out standard conditions which may include excluding oxygen, and mechanical stirring. Those skilled in the art will readily identify and implement other alternative core materials and methods to implement them.

Once the cores or scaffolds have been prepared, the body of the particle, which may be or comprise a metal oxide can be prepared and deposited on the core. In particular embodiments the metal oxide may be an iron oxide but those skilled in the art will readily identify and implement alternative suitable materials. FIGS. 6C and D respectively are TEM images of PS/Fe₃O₄ nanostructures.

In one variant of the embodiment the metal oxide deposition may be achieved as follows: A suitable metal compound which may be a chloride salt, may be dissolved in a suitable solvent and mixed with the core particles. Where the metal is iron then the compound may be FeCl₃, and the solvent may be ethylene glycol aqueous solution with added hexamethylene tetramine. The mixture may be treated, for example by heating or other suitable adjustments, all of which will be readily identified and implemented by those skilled in the art, in view of the metal oxide in question and the desired parameters. Where the metal is iron then mixture may be heated to 80° C. or other suitable temperature, for a desired time period, with the exclusion of oxygen if desired, so as to precipitate (PS/Fe₃O₄) particles. The PS core can then be removed by calcination at a suitable temperature, under suitable conditions. For example calcination may be carried out at about 400-500° C. for about 3 h in the presence of nitrogen gas. FIG. 7 shows iron oxide nanoparticles (nanoshells) obtained using the disclosed methods.

It will be understood that the materials used for the core, and for the particles, the methods for constructing the core, the methods for depositing the particle body about the core and the methods for degrading the core to liberate hollow or porous nanoparticles, will all be readily adapted by those skilled in the art to suit specific requirements. In embodiments where the newly-synthesized nanoparticles are iron oxide nanoparticles, it has been found that the nanoparticles may be nearly monodispersed. In one embodiment the average or median size of the nanoparticles may be controlled as between about 100 nm and about 200 nm in diameter or between about 125 nm and about 175 nm in diameter, or between about 135 nm and 165 nm, or between about 140 nm and about 160 nm, or between about 145 nm and about 155 nm and in particular embodiments may have a diameter of about '50 nm

In a further aspect of the embodiment, the nanoparticles may be aggregated with a matrix to form embolisation compositions with which are of a desired size. In particular forms of the embodiment the embolisation compositions may be about 1.5 mm in diameter or may be larger or smaller, and may be substantially spherical solid or semisolid embolisation compositions. In variants of the embodiment, the matrix may be or may comprise PVA and may have any desired shape or consistency

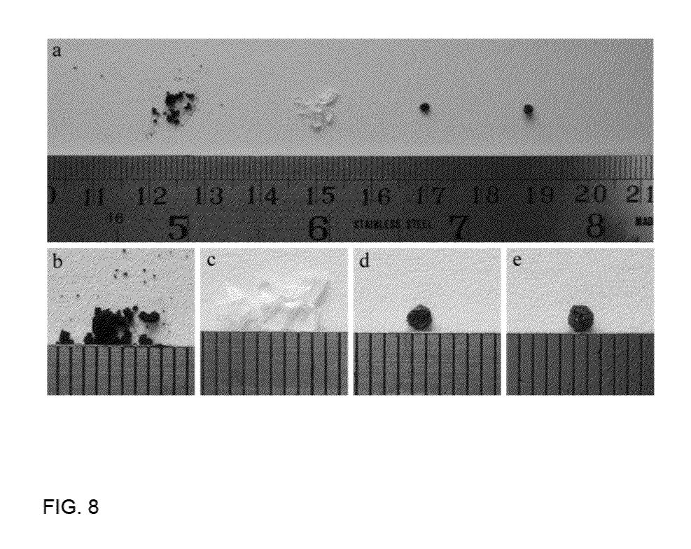

FIG. 8 shows examples of embolisation compositions according to an embodiment. FIG. 8A is a photograph showing from left to right hollow porous iron oxide nanoshells, PVA matrix alone, and two examples of iron oxide shell/PVA compositions (embolisation compositions). The illustrated examples of embolisation compositions are iron oxide nanoshell/PVA (1:0.5 ratio) composite (1.5 mm sphere) on the left, and iron oxide nanoshell/PVA (1:1.5 ratio) composite (1.5 mm sphere) on the right. FIGS. 8B through E are higher magnification photographs and show, respectively, iron oxide nanomaterial (8B); PVA (8C); prototype iron oxide nanomaterial/PVA composite (1.5 mm, 1:0.5 nanoshell/PVA ratio) (8D) and iron oxide nanoshell/PVA composite (1.5 mm sphere, 1:1.5 nanoshell/PVA ratio) (8E). It will be understood that by adjustments to the manufacturing procedure, in variants of the method the surface of iron oxide nanoshell/PVA composite may be further smoothed. The ratio between iron oxide nanoshell and PVA will dictate the rates of breakdown of the matrix and of controlled release of the nanoparticles and/or the therapeutic agent held by the nanoparticles. In embodiments a higher percentage PVA content in the matrix may lower the rate of release of therapeutic agents held within the nanoparticles and vice versa, but the properties of particular compositions will be determined by routine testing and with the common understanding of the properties of suitable matrix materials, as will be understood by those skilled in that art.

Once synthesised, nanoparticles may be ultrasonically dispersed, for example in an ethanol/water mixture, and following this a desired amount of pre-swelled PVA may be added to form the embolisation compositions. The resulting embolisation composition may be dried under vacuum.

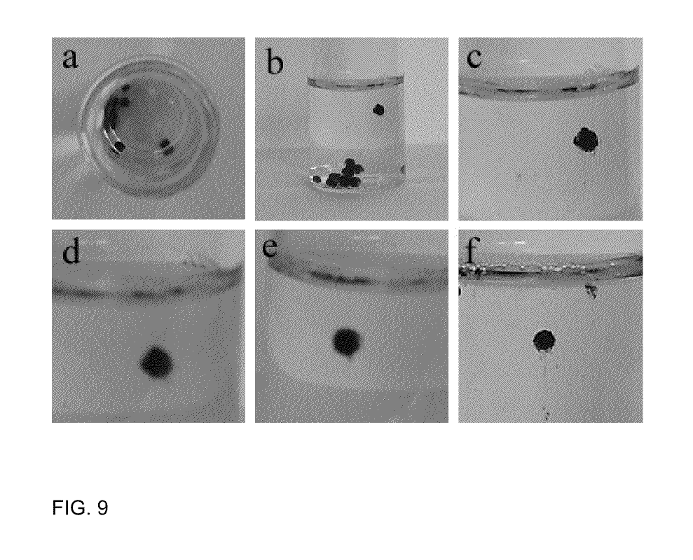

The ratio between nanoparticles and matrix, and the properties of matrix, will dictate the rates of controlled release of a therapeutic agent. That is, where an embolisation composition comprises iron oxide particles and PVA matrix, then a higher relative PVA content may lowers the rate of release of the therapeutic agent and vice versa. It has also been found that in some embodiments, the use of a PVA with higher hydrolyzation level such as PVA98 (which may be about 98-99% hydrolyzed) reduces the speed of dissolution of the matrix, however the less hydrolised PVA such as PVA80 (about 80% hydrolyzed) increases the speed. In particular embodiments a mixture of PVA98 and PVA80 is used to control the matrix dissolution rate within suitable parameters for therapeutic purposes. The ability of suitable matrix materials to restrict the release of an agent stored within embedded nanoparticles is shown particularly in FIG. 9. FIG. 9a, 9b, 9c which are photographs of iron oxide nanoshell/PVA composite spheres in media at different magnifications. In FIG. 9d, d, f: one iron oxide nanoshell/PVA composite sphere is shown attached to the wall of a vial at different time points under gravity (9d 1 hr, 9e 4 hr, 9f 6 hr). As will be seen there is a progressive dissociation of iron oxide nanoparticles from the PVA matrix. The example shown in FIG. 9d, 9e, 9f uses a mixture of PVA98 and PVA80 of about 1:2 with a molecular weight between about 9,000 and 50,000 and the ratio for iron oxide to PVA is about 1:1.5. It has been found that the use of PVA with higher hydrolyzed rate can slow down the dissociation process. This adjustment of matrix properties can thus adjust the rate at which an agent, such as a therapeutic agent, is released from the embedded nanoparticles and thus from an embolisation composition.

In one example, about 6 mg of the therapeutic agent is dissolved in about 0.6 ml of water. Then, about 30 mg of iron oxide nanoshells are added and the solution. The water from the solution is evaporated in vacuum. The drug loaded iron oxide nanoparticles are dispersed into the PVA solution, and the mixture is dried in vacuum to for the composite material.

In embodiments, the embolisation compositions comprising nanoparticles and encapsulating matrix, may be cut or shaped to form desired shapes and sizes in a variety of conventional ways. In embodiments the embolisation compositions may be cut to be spherical bodies which may be of any desired size but may be about 1.5 mm diameter or may be up to or greater than about 0.1 mm, 0.2 mm, 0.3 mm, 0.4 mm, 0.5 mm, 1.0 mm, 1.5 mm, 2.0 mm, 2.5 mm, 3.0 mm, 3.5 mm, 4.0 mm or more than 4.0 mm in diameter or their diameter may be in a range whose lower limit is about 0.5 mm, 1.0 mm, 1.5 mm, 2.0 mm, 2.5 mm, 3.0 mm, 3.5 mm, 4.0 mm and whose upper limit may be about 0 mm, 1.5 mm, 2.0 mm, 2.5 mm, 3.0 mm, 3.5 mm, 4.0 mm or 4.5 mm, or may be between about 0.5 and about 3 mm, between about 1.0 mm and 2.5 mm, or between about 1.0 mm and about 1.5 nm In particular embodiments the embolisation compositions may be between about 0.2 mm and about 2 mm in diameter. The embolisation compositions may be of any desired shape and may be spherical, ovoid, flattened, cubical, elongate, conical, regular, irregular or any desired shape and any desired size. Those skilled in the art will readily select suitable shapes and sizes for particular applications and will readily understand how, with routine testing and adjustments, such parameters may be adjusted.

While changes to matrix thickness and concentration may be used to control the rate of release of an agent, such as a therapeutic agent, the embolisation compositions may also be coated with gelatin or any other suitable material having desired properties. Thus for example an embolisation composition may be coated with additional layers of matrix material, or with gelatin, or with other materials suitable to modulate the time over which the embolisation composition begins to break down or to otherwise modify the surface or other chemical or physical properties of the embolisation composition.

### Preparation and Chemical Release of Nanoparticle/Matrix/Drug Composite Materials

In an embodiment, synthesised nanoparticles may be loaded with chemicals, which may be therapeutic agents and may be anti-cancer drugs such as taxol, doxorubicine, cisplatin or other suitable therapeutic agents, all of which will be readily apparent to those skilled in the art. The nanoparticles may be incubated with the desired chemical in a solvent and then mixed with a solution of swelled matrix material. In one version of an embodiment the matrix material may be or comprise PVA. To trap a therapeutic agent in the nanoparticles, the therapeutic agent and the nanoparticles may be modified in a variety of ways all of which will be readily apparent to those skilled in the art. By way of example, molecules of therapeutic agent may combined or associated with other carrier molecules or excipients having desired properties, for example increased charge, hydrophilicity, or hydrophobicity, all as necessary or desirable to fine-tune the encapsulation and release rate. Similarly the interior surfaces of the nanoparticles, and/or any nanopores therein, may be modified or treated to confer desirable surface properties such as modified hydrophilicity, hydrophobicity, or to bear specific chemical groups suitable to associate with particular therapeutic agents. One form of possible modification is treatment with silylation reagents. In particular, nanoparticles may be modified so as to be more hydrophilic, to better hold hydrophilic therapeutic agents and particles may be treated to make them more hydrophobic where they are to be used to carry more hydrophobic agents.

### Alternative Embodiments and Variants

In alternative embodiment there is disclosed a method for inhibiting growth of a cancer, the method comprising inhibiting the blood supply to the cancer with a degradable embolisation composition, the embolisation composition comprising a degradable matrix, a plurality of magnetic nanoparticles, and a therapeutically effective amount of a therapeutic agent. The method may further comprise introducing the embolisation composition into the blood supply using a catheter. The method is carried out in the same manner as the other embodiments disclosed herein.

In embodiments, embolisation compositions comprising nanoparticles and matrix may be sized between about 0.5 mm and 4 mm in diameter and may be about 1-1.5 mm or about 1.5 mm to 2 mm, or may be between about 1 mm and 2 mm in diameter. They may be delivered to the feeding arteries of tumors via a selective catheter. Upon delivery, these embolisation compositions may block or obstruct small arteries or arterioles feeding a tumour so that the tumor is deprived of blood supply or so that blood supply to the tumour is reduced. It will be readily apparent to those skilled in the art that in alternative embodiments the embolisation compositions may be larger or smaller to suit specific requirements.

With modifications that will be readily understood by those skilled in the art, the methods and reagents disclosed can also be used for magnetically controlled hyperthermia treatment, by using the applied magnetic field to raise the temperature of the nanoparticles, or may be used for treatment of a range of other conditions.

It is also contemplated that in embodiments that the nanoparticles containing therapeutic agents may be delivered to a patient's body intravenously. The choice between the various methods for delivery of nanoparticles and embolisation compositions will be readily understood and selected amongst by those skilled in the art.

In a further embodiment there is disclosed a drug delivery system comprising a hollow magnetic nanoparticle, or a plurality of such nanoparticles, which may be formed into an embolisation composition and may comprise polymers or biomolecules or other materials for the controlled release of a therapeutic agent. In embodiments the nature of such controlled release may be adjusted by adjusting the relative content of the nanoparticles and the matrix, or by modifying the properties of the nanoparticles or of the matrix.

In a further embodiment there is disclosed an embolisation or chemoembolisation system, which may comprise one or more embolisation compositions, which may use the nanoparticles of embodiments, and may be used for the delivery of therapeutic agents which may be anti-cancer agents. In a further embodiment compositions of other embodiments are used to locally deliver a therapeutic agent.

### EXAMPLES

The following examples are provided by way of illustration only and are not limiting:

### DETAILED DESCRIPTION

### Preparation of the Iron Oxide Nanoshell Nanoparticles

Mesoporous iron oxide nanoshells with a diameter of 200 nm were prepared by using a polystyrene acrylate templating method as shown in FIG. 5. Briefly, negatively charged carboxyl-stabilized polystyrene (PS) latex with an average diameter of 200 nm (FIG. 5A) was prepared as also shown in FIGS. 6A, 6B. Styrene and acrylic acid monomer (100:2 w/w) were copolymerized in the emulsion using sodium dodecyl sulfate as the surfactant and ammonium persulfate as the initiator. The reaction was carried out in nitrogen at 70° C. with mechanical stirring at a speed of 350 rpm. The PS particles were purified by three centrifugation and dispersion cycles in water. Then FeCl₃ (1 g) was added in ethylene glycol aqueous solution (5 ml) in the presence of PS nanoparticles and hexamethylene tetramine (0.4 g). The mixture was heated to 80° C. for 3 h under nitrogen. The precipitate (PS/Fe₃O₄) (FIG. 5B, C, E) was washed and dried at 50° C. and is illustrated in FIGS. 6C, 6D. It will be seen that the thickness of the metal oxide nanoparticle may be adjusted by changing the deposition time (FIG. 5B, C, E). The PS core was removed by calcination at 400-500° C. for 3 h in the absence or substantial absence of oxygen. This may be achieved by carrying out the calcination under an atmosphere of nitrogen gas, and mesoporous iron oxide nanoshells of a desired thickness were obtained and are shown in FIG. 7, and as FIGS. 5D and F.

In particular examples, the as-synthesized iron oxide nanoshells may be nearly monodispersed. Their average sizes may be controlled in the range of approximately 150-200 nm and were successfully characterized by TEM and are shown in FIG. 7. These iron oxide nanoparticles/nanoshells may be used for drug encapsulation and PVA incorporation.

The iron oxide nanoshells were incorporated with PVA to form substantially uniform 1.5 mm substantially spherical solids as the chemoembolisation compositions. By way of example, iron oxide nanoshell/PVA (1:0.5) composite (1.5 mm sphere) and iron oxide nanoshell/PVA (1:1.5) composite (1.5 mm sphere) were prepared and are shown in FIG. 8. Generally, iron oxide nanoshells were ultrasonically dispersed in ethanol/water mixture for at least 30 min, followed by the addition of a chosen amount of PVA that is pre-swelled in water. The water from the solution is evaporated in vacuum. The drug loaded iron oxide nanoparticles are dispersed into the PVA solution, and the mixture is dried in vacuum to for the composite material. The ratio between iron oxide nanoshell and PVA will dictate the rates of controlled release. That is, higher % PVA content would be expected to lower the rate of release and vice versa, but the properties of particular compositions will be determined by routine testing as will be understood by those skilled in that art. In variants the mixture of PVA98 and PVA80 can be used to control the reasonable matrix dissolution rate. PVA with a higher hydrolysation level of PVA such as PVA98 which may be about 98-99% hydrolyzed, may reduce the speed of dissolution of the matrix, however a lower hydrolysation level of PVA such as PVA80 which may be about 80% hydrolyzed, may increase the dissolution rate.

### Preparation and Chemical Release Test of the Iron Oxide Nanoshell/PVA/Drug Composite Materials

The iron oxide nanoshells were incubated with anti-cancer drug molecules (Taxol, doxorubicine) in a solvent and then incorporated with a solution of swelled PVA. As shown in FIG. 9, it is believed that the PVA may block the pores in the nanoparticle to prevent release of the therapeutic agent. The mixing of different amounts of each substance was tested. A preferred formulation was investigated and obtained. Briefly, substrate (drug molecules or other organic compounds, typically about 0.6 mg) was dispersed in water (0.6 ml). Then, iron oxide nanoshells (30 mg) were added. The water from the solution was evaporated by freeze-drying. 0.3 ml of PVA98 (50 mg/ml) was added to the iron oxide which had been adsorbed with therapeutic agent, and then freeze-drying was performed to evaporated the water and form therapeutic agent/iron oxide/PVA98 composite material. This composite material was suspended into 0.6 ml of PVA80 (50 mg/ml) solution. This suspension was dropped into liquid nitrogen, and the frozen particles were applied with freeze-drying to form the final iron oxide/therapeutic agent/PVA composite material.

The as-prepared composite was shaped to form spheres (or other shapes as desired, e.g., cubes) with a diameter of about 1.5 mm (or other selected sizes as desired). These composite materials were readily used for chemoembolisation. FIGS. 9a, 9b, 9c: are photographs of the iron oxide nanoshell/PVA composite spheres in media at different magnifications. FIGS. 9d, 9e and 9f: show one iron oxide nanoshell/PVA composite sphere attached to the wall of a vial at different time points under gravity. FIGS. 9d, 9e and 9f show the 1, 4, and 6 hour time points respectively. Dissociation of the iron oxide nanostructure from PVA matrix can be seen over the course of these timepoints. The PVA used was a 1:2 mixture of PVA98 to PVA80 and the ratio of iron oxide to PVA was about 1:1.5.

The embodiments and examples presented herein are illustrative of the general nature of the subject matter claimed and are not limiting. It will be understood by those skilled in the art how these embodiments can be readily modified and/or adapted for various applications and in various ways without departing from the spirit and scope of the subject matter disclosed claimed. The claims hereof are to be understood to include without limitation all alternative embodiments and equivalents of the subject matter hereof. Phrases, words and terms employed herein are illustrative and are not limiting. Where permissible by law, all references cited herein are incorporated by reference in their entirety. It will be appreciated that any aspects of the different embodiments disclosed herein may be combined in a range of possible alternative embodiments, and alternative combinations of features, all of which varied combinations of features are to be understood to form a part of the subject matter claimed.

What is claimed is:

1. An embolization composition comprising:
a biodegradable matrix encapsulating at least one nanoparticle, wherein degradation of the matrix releases the nanoparticle.

2. The embolisation composition according to claim 1 wherein the nanoparticle is hollow.

3. The embolisation composition according to claim 2 wherein the nanoparticle is a superparamagnetic porous nanoparticle.

4. The embolisation composition according to claim 2 wherein said nanoparticle contains a therapeutically effective amount of a therapeutic agent.

5. The embolisation composition according to claim 1 wherein the matrix comprises PVA.

6. The embolisation composition according to claim 4 wherein the therapeutic agent is an anticancer agent.

7. The embolisation composition according to claim 1 wherein the nanoparticle is coated with:
a separating agent; or
a targeting agent; or
a separating agent and a targeting agent.

8. A method of selectively obstructing blood flow in a blood vessel in a patient, the method comprising the steps of:
administering to the patient an embolization composition according to claim 1 for purposively inducing an embolism in said patient blood vessel.

9. A method for delivering a therapeutic agent to a patient, the method comprising the steps of:
administering to the patient an embolisation composition according to claim 1 that contains the therapeutic agent; and
releasing the therapeutic agent.

10. The method according to claim 9 wherein the method further comprises introducing said embolisation composition into a blood vessel supplying blood to a target region.

11. The method according to claim 9 further comprising the step of applying a magnetic field to position said embolisation composition.

12. The method according to claim 10 wherein said target region comprises a tumor.

13. A method for making an embolisation composition, the method comprising the step of embedding at least one nanoparticle in a degradable matrix to thereby form the embolisation composition.

14. The method according to claim 13 wherein said nanoparticle contains a therapeutic agent.

15. The method according to claim 9 wherein said nanoparticle is a hollow, superparamagnetic nanoparticle.

16. The method according to claim 14 wherein when said matrix suppresses the release of said therapeutic agent.

17. The method according to claim 14 wherein the therapeutic agent is an anticancer agent.

18. The method according to claim 13 wherein the nanoparticle is coated with:
a separating agent; or
a targeting agent; or
a separating agent and a targeting agent.

19. A method for inhibiting growth of a cancer, the method comprising the step of inhibiting the blood supply to said cancer with a degradable embolisation composition
said embolisation composition comprising a degradable matrix, a plurality of magnetic nanoparticles, and a therapeutically effective amount of an anticancer agent.

20. The method according to claim 19 wherein said nanoparticles are embedded in said matrix and carry said therapeutic agent, and wherein said method further comprises the steps of:
allowing ones of said nanoparticles to escape from the blood vessel carrying said blood supply; and
releasing the anticancer agent from said nanoparticles.
